(19) United States
(12) Patent Application Publication
(10) Pub. No.: US 2021/0186369 A1
(43) Pub. Date: Jun. 24, 2021

(54) BREATH DETECTION DEVICE AND METHOD THEREOF

(71) Applicant: WISTRON NEWEB CORPORATION, HSINCHU (TW)

(72) Inventors: YA-PING WEI, HSINCHU (TW); HOREN CHEN, HSINCHU (TW)

(21) Appl. No.: 16/718,314

(22) Filed: Dec. 18, 2019

(51) Int. Cl. A61B 5/08; A61B 5/00; A61B 5/05; G01S 13/00; G01S 7/41

(52) U.S. Cl. CPC A61B 5/0816; A61B 5/7203; G01S 7/415; A61B 5/7278; G01S 13/003; A61B 5/05

(57) ABSTRACT

A breath detection device and method thereof are provided. The system includes a transmitter, a receiver, a processor and a memory. The transmitter transmits a signal, and the receiver receives the signal from a multipath channel being impacted by breathing of a living being. The processor is configured to: extract CSI within a first time interval for the multipath channel from the at least one wireless signal; calculate one or more breathing rates based on the CSI within the first time interval; delete one or more breathing rates exceeding a predetermined range from the one or more breath breathing rates; group the remaining breathing rates to obtain one or more breathing rate groups; select the breathing rate group having largest quantity; and take the breathing rate of the selected breathing rate group as an estimated breathing rate.

### FIELD OF THE DISCLOSURE

The present disclosure relates to a breath detection device and method thereof, and more particularly to a breath detection device and method thereof capable of ignoring interference to subcarriers and improving an accuracy of breath detection.

### BACKGROUND OF THE DISCLOSURE

The existing breath detection technology uses channel state information (CSI) obtained from RF signals to detect the number of breaths, thus replacing traditional wearable devices or monitoring devices. This technology allows subjects to retain privacy while not being bound by detection sites. The subjects are only required to stay in a wireless communication location, and after receiving the RF signal, the breath detection can be performed anytime and anywhere.

Specifically, in the above-mentioned breath detection technology, a CSI acquisition technique is adopted, which extracts the amplitude or phase of a subcarrier in the CSI, then filters noise and outliers, sets a window function and calculates the number of peaks to predict the number of breaths per minute.

Since each subcarrier in the CSI is interfered to different extents, a better state of the subcarrier must be selected beforehand to predict the number of breaths from its regular respiratory cycle phenomenon, thus causing an increase in detection cost.

### SUMMARY OF THE DISCLOSURE

In response to the above-referenced technical inadequacies, the present disclosure provides a breath detection device and method thereof.

In one aspect, the present disclosure provides a breath detection device, which includes a receiver, a processor and a memory. The receiver is configured to receive at least one signal from a multipath channel being impacted by breathing of at least one living being, and the memory communicatively coupled to the processor. The processor is configured to: extract channel state information (CSI) within a first time interval for the multipath channel from the at least one wireless signal; calculate one or more breathing rates based on the CSI within the first time interval; delete one or more breathing rates exceeding a predetermined range from the one or more breath breathing rates; group the remaining breathing rates to obtain one or more breathing rate groups; select the breathing rate group having largest quantity; and take the breathing rate of the selected breathing rate group as an estimated breathing rate.

In one aspect, the present disclosure provides a breath detection method, which includes the following steps: configuring a transmitter to transmit at least one signal; configuring a receiver to receive the at least one signal from a multipath channel being impacted by breathing of at least one living being; configuring a processor to: extract channel state information (CSI) within a first time interval for the multipath channel from the at least one wireless signal; calculate one or more breathing rates based on the CSI within the first time interval; delete one or more breathing rates exceeding a predetermined range from the one or more breath breathing rates; group the remaining breathing rates to obtain one or more breathing rate groups; select the breathing rate group having largest quantity; and take the breathing rate of the selected breathing rate group as an estimated breathing rate.

Therefore, the breath detection device and method thereof provided by the present disclosure can predict the one or more breathing rates by considering all subcarriers from the obtained CSI in the obtained parent set, analyze the results of the reaction of the subcarriers in the indoor environment according to the statistical distribution ratio, and select the breathing rates having the highest proportion to obtain a reasonable breathing rate.

Furthermore, the breath detection device and method thereof provided by the present disclosure can reduce breathing detection costs without selecting a better state of the subcarrier beforehand, so as to ignore interference to subcarriers and improve an accuracy of breath detection.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The present disclosure will become more fully understood from the following detailed description and accompanying drawings.

FIG. 1 shows a block diagram of a breath detection system in accordance with an exemplary embodiment of the present disclosure.

FIG. 2 is a flow chart showing a general process of a breath detection method according to an embodiment of the present disclosure.

FIGS. 3A and 3B show block diagrams of a breath detection device in accordance with another exemplary embodiment of the present disclosure.

FIGS. 4A and 4B are flow charts showing other processes of a breath detection method according to another embodiment of the present disclosure.

FIG. 5 is a schematic time line showing breathing rate estimations with times according to an embodiment of the present disclosure.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a”, “an”, and “the” includes plural reference, and the meaning of “in” includes “in” and “on”. Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first”, “second” or “third” can be used to describe various components, signals or the like, which are for distinguishing one component/signal from another one only, and are not intended to, nor should be construed to impose any substantive limitations on the components, signals or the like.

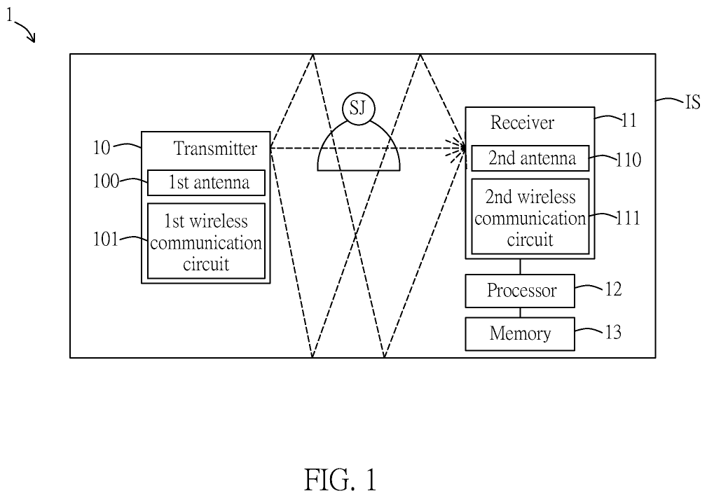

Reference is made to FIG. 1, FIG. 1 shows a block diagram of a breath detection system in accordance with an exemplary embodiment of the present disclosure. As shown, the present disclosure provides a breath detection system 1, which includes a transmitter 10, a receiver 11, a processor 12 and a memory 13, and the memory 13 is communicatively coupled to the processor 12. The transmitter 10 can include a first antenna 100 and a first wireless communication circuit 101 for controlling a transmitting direction of the first antenna 100. The first wireless communication circuit 101 can support plural of protocols and may be used to transmit wireless signals having different working frequencies. Furthermore, the protocols may be wireless communication standard, such as, IEEE 802.11, 3G/4G/5G standards.

Similarly, the receiver 11 can include a second antenna 110 and a second wireless communication circuit 111 for controlling a transmitting direction of the second antenna 110. Similarly, the second wireless communication circuit 111 can support plural of protocols corresponding to the transmitter 10 and may be used to transmit wireless signals having different working frequencies. Furthermore, the protocols may be wireless communication standard, such as, IEEE 802.11, 3G/4G/5G standards.

In one embodiment, the transmitter 10 and the receiver 11 can communicate with each other through a network that is at least one of: Internet, an Internet-protocol network, and another multiple access network; and the receiver is associated with a physical layer of at least one of: a wireless PAN, IEEE 802.15.1 (Bluetooth), a wireless LAN, IEEE 802.11 (Wi-Fi), a wireless MAN, IEEE 802.16 (WiMax), WiBro, HiperMAN, mobile WAN, GSM, GPRS, EDGE, HSCSD, iDEN, D-AMPS, IS-95, PDC, CSD, PHS, WiDEN, CDMA2000, UMTS, 3GSM, CDMA, TDMA, FDMA, W-CDMA, HSDPA, W-CDMA, FOMA, 1×EV-DO, IS-856, TD-SCDMA, GAN, UMA, HSUPA, LTE, 2.5G, 3G, 3.5G, 3.9G, 4G, 5G, 6G, 7G and beyond, another wireless system and another mobile system.

In the present embodiment, the disclosed breath detection system 1 can operate based on Wi-Fi networks in an indoor space IS, and the Wi-Fi networks are easily and cheaply available. Therefore, the system can be deployed, managed and maintained easily and can be obtained at low cost. The disclosed system can further operate in both line-of-sight (LOS) and non-line-of-sight (NLOS) propagation conditions, and possibly for through-the-wall detection as well. A LOS environment means there is a direct LOS path between the transmitter 10 or the receiver 11 and a subject SJ (a living being such as human being or animal) to be detected or whose breathing rate is to be tested.

In contrast, in a NLOS environment, there are some blockages, e.g. walls, between the device and the test subject, such that no light can directly pass through the straight path between the device and the potential subject.

In the present embodiment, the transmitter 10 is configured to transmit at least one signal, and the receiver 11 is configured to receive the at least one signal from a multipath channel being impacted by breathing of at least one living being, that is, the subject SJ. It should be noted that the at least one signal can be transmitted in a bandwidth of 80 MHz

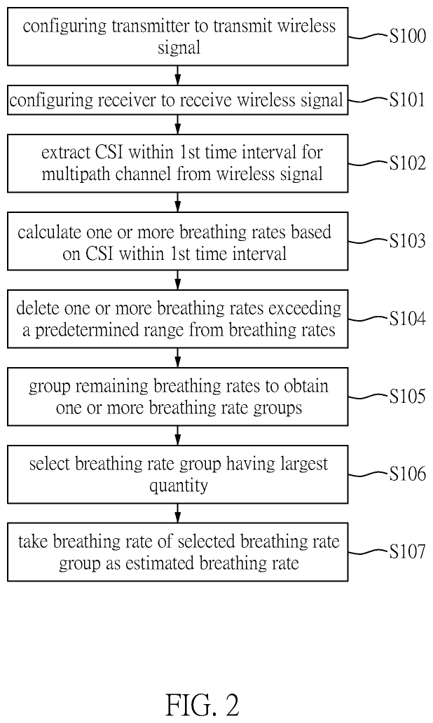

Reference is made to FIG. 2, which is a flow chart showing a general process of a breath detection method according to an embodiment of the present disclosure. As shown, the breath detection method includes at least the following steps:

Step S100: configuring the transmitter 10 to transmit at least one signal. In the present step, the transmitter 10 can transmit radio signals, such as a pulse or a pseudo random sequence through a multipath channel.

Step S101: configuring the receiver 11 to receive the at least one signal from a multipath channel being impacted by breathing of the subject SJ. In the present step, the receiver 11 can receive the signals from the multipath channel that are impacted by the breathing of the subject SJ.

Step S102: configuring the processor 12 to extract channel state information (CSI) within a first time interval for the multipath channel from the at least one wireless signal. The CSI are extracted in the present step from the received radio signals using channel estimation, and the first time interval can be larger than a duration of a breath taken by a healthy adult, for example, 6 to 18 seconds. In detail, human breathings are usually taken once every 2 seconds to 6 seconds, and thus the first time interval provided here uses the max value of 6 seconds. In order to obtain a sufficient amount of data, 18 seconds are preferably used for an example, and the present is not limited thereto.

In more detail, the processor 12 can further process the CSI to obtain processed CSI, for example, each component in the CSI can be composed by an amplitude and a phase of a sub-carrier, and when multiple subcarriers arrive at the receiver 11 along the multipath channel, each sub-carrier may have its own amplitude and phase. Therefore, the processor 12 can process the CSI to obtain amplitudes of the sub-carriers as the processed CSI.

Step S103: configuring the processor 12 to calculate one or more breathing rates based on the CSI within the first time interval. In detail, a periodogram is an algorithm which can be utilized to predict the number of cycles of the amplitude of each subcarrier. It should be realized that the periodogram is an estimate of the spectral density of a signal in signal process, and the periodogram can be further used for examining the amplitude versus frequency characteristics of finite impulse response (FIR) filters and window functions. Moreover, the number of breaths per minute can be defined as a breathing rate. In this regard, the number of breaths can be obtained according to the amplitude of each of the subcarriers. After one or more breathing rates are calculated, the one or more breathing rates can serve as a set of breathing rate candidates.

For example, given ten subcarriers are included in the processed CSI, the set of breathing rate candidates is shown in the following Table I:

TABLE I

| Subcarrier index | 1 | 2 | 3 | 4 | 5 | 6 | 7 | 8 | 9 | 10 |
|---|---|---|---|---|---|---|---|---|---|---|
| Breathing rates | 15 | 97 | 97 | 1 | 15 | 15 | 15 | 15 | 10 | 10 |
| candidates | | | | | | | | | | |
| (times/minute) | | | | | | | | | | |

Step S104: configuring the processor to delete one or more breathing rates exceeding a predetermined range from the one or more breathing rates.

In more detail, given the predetermined range is from breathing rates of 10 times per minute to 30 times per minute, breathing rate candidates exceeding the predetermined range are regarded as outliers and should be deleted (by denoting strikethroughs), as shown in the following Table II:

TABLE II

| Subcarrier index | 1 | [symbol] | [symbol] | 4 | 5 | 6 | 7 | 8 | 9 | 10 |
|---|---|---|---|---|---|---|---|---|---|---|
| Breathing rates | 15 | [symbol] | [symbol] | [symbol] | 15 | 15 | 15 | 15 | 10 | 10 |
| candidates | | | | | | | | | | |
| (times/minute) | | | | | | | | | | |

Step S105: configuring the processor 12 to group the remaining breathing rates to obtain one or more breathing rate groups. In this example, the remaining breathing rates are 10(times/minute) and 15(times/minute), and the processor 12 is configured to group the remaining breathing rates to obtain two breathing rate groups. 10(times/minute) breathing rate group includes two breathing rate candidates, which indicates that two subcarriers reacting a breathing rate of 10 times per minute, and 15(times/minute) breathing rate group includes five breathing rate candidates, which indicates that five subcarriers reacting a breathing rate of 15 times per minute.

Step S106: configuring the processor 12 to select the breathing rate group having largest quantity. For example, the quantity can be the number of subcarriers, the 15(times/minute) breathing rate group has largest quantity of five subcarriers, and thus the 15(times/minute) breathing rate group is selected.

Step S107: configuring the processor 12 to take the breathing rate of the selected breathing rate group as an estimated breathing rate. For example, since the 15(times/minute) breathing rate group is selected, the breathing rate of 15 times per minute is taken as the estimated breathing rate.

Specifically, the disclosed system predicts the one or more breathing rates by considering all subcarriers from the obtained CSI in the obtained parent set, analyzes the results of the reaction of the subcarriers in the indoor environment according to the statistical distribution ratio, and select the breathing rates having the highest proportion to obtain a reasonable breathing rate. Therefore, the breath detection system and method thereof provided by the present disclosure can reduce breathing detection costs without selecting a better state of the subcarrier beforehand, so as to ignore interference to subcarriers and improve an accuracy of breath detection.

For example, for humans, the predetermined range for a healthy adult at rest can range from a lower breathing rate to a higher breathing rate, such as 2 to 6 seconds per breath, and the first time interval should be larger than the higher breathing rate, such as 6 to 18 seconds mentioned above.

Furthermore, the disclosed system can employ a number of wireless transmitters and receivers working together to implement the breath detection functionality. One possible embodiment of the disclosed system is to use Wi-Fi wireless transceivers. But the disclosed system is not limited to such devices. It can be used with any wireless devices that can provide CSI as a part of its operation (e.g. Bluetooth, 3GPP LTE transceivers, or any custom-designed non-standard-compliant wireless transceiver).

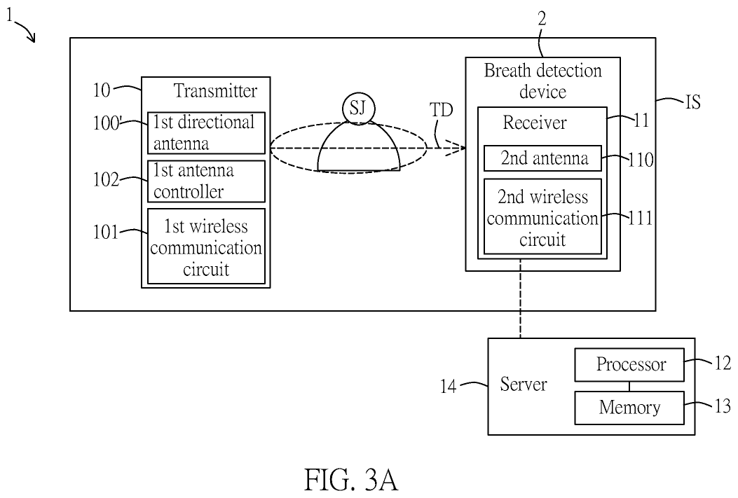

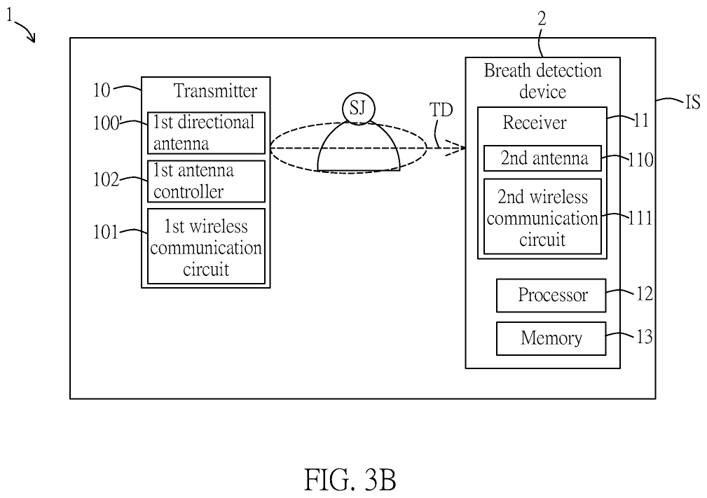

Reference is further made to FIGS. 3A and 3B, which show block diagrams of a breath detection device in accordance with another exemplary embodiment of the present disclosure.

In the present embodiment, like reference numerals denote to the like components, and the repeated description are omitted.

As shown in FIG. 3A, the breath detection system 1 further includes a server 14, which is communicatively coupled to the receiver 11, the server includes the processor 12 and the memory 13, and the receiver 11 is configured to transmit data of the received at least one wireless signal to the server 14.

The server 14 takes the data of the received at least one wireless signal as input, extracts CSI and features relevant to breathing (or other vital signs having a periodic pattern) that describe the wireless channel behavior between the transmitter 10 and the receiver 11, and generate outputs that quantify the breathing rates of the subject SJ being in the vicinity of the wireless transceivers 10.

Furthermore, the transmitter 10 further includes a first directional antenna 100′ and a first antenna controller 102, the first antenna controller 102 is configured to control the directional antenna 100′ to transmit the at least one wireless signal in a transmitting direction TD toward the living being. In more detail, the directional antenna 100′ is configured to form a radiation pattern having a primary beam towards to the transmitting direction TD.

Since the energy of the signal transmitted through a detection area associated to the subject SJ is concentrated, the accuracy of the breath detection can be relatively improved for the subject SJ to change the breathing posture, such as lying down, lying down, sleeping sideways, curling the body, and the like.

In FIG. 3B, a breath detection device 2 is provided. The breath detection device 2 includes the receiver 11, the processor 12 and the memory 13, the operations thereof are mentioned in the previous embodiment, and the repeated descriptions are omitted accordingly.

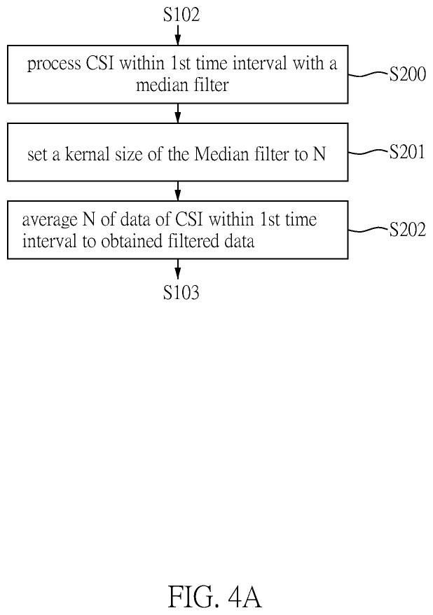

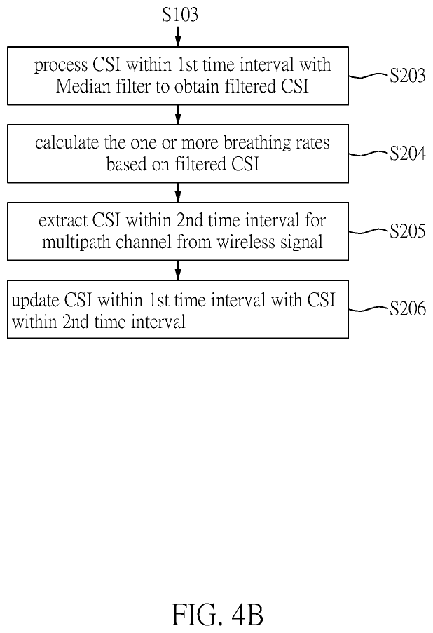

Reference is further made to FIGS. 4A and 4B, which are flow charts showing other processes of a breath detection method according to another embodiment of the present disclosure.

In the present embodiment, a median filter is further utilized to process raw amplitude data of the CSI. The median filter runs through the data of the CSI obtained from the received signal entry by entry, replacing each entry with the median of neighboring entries. The pattern of neighbors is called the “window”, which slides, entry by entry, over the entire signal. The size of the “window” can be defined as a kernel size of the median filter.

As shown in FIG. 4A, the breath detection method provided by the present disclosure can further include the following steps after the step S102:

Step S200: configuring the processor 12 to process the CSI within the first time interval with a median filter.

Step S201: configuring the processor to set a kernal size of the Median filter to N. It should be noted that N is an odd number larger than 2.

Step S202: configuring the processor to average N of data of the CSI within the first time interval to obtained filtered data.

After the step S202, the steps S103 can be performed.

Reference is made to FIG. 4B. In another preferred embodiment, if the one or more breathing rates are unable to be calculated based on the CSI within the first time interval in the step S103, a step S203 is performed.

Step S203: configuring the processor 12 to process the CSI within the first time interval with Median filter to obtain a filtered CSI

Step S204: configuring the processor 12 to calculate the one or more breathing rates based on the filtered CSI.

After the one or more breathing rates based on the filtered CSI is calculated, the method proceeds to the following steps:

Step S205: configuring the processor 12 to extract CSI within a second time interval for the multipath channel from the at least one wireless signal, wherein the second time interval is after the first time interval.

Step S206: configuring the processor 12 to update the CSI within the first time interval with the CSI within the second time interval.

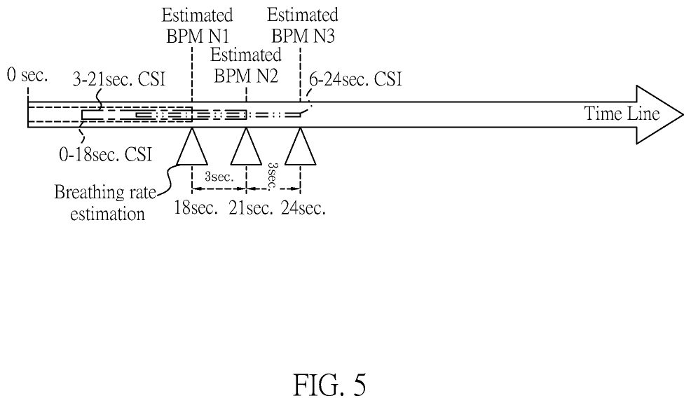

Reference can be further made to FIG. 5, which is a schematic time line showing breathing rate estimations with times according to an embodiment of the present disclosure. In detail, data collecting and data calculating for obtaining the breathing rate is continuous, for example, after the first time interval of 18 seconds, CSI within the second time interval of 3 seconds after the first interval are obtained for estimating a first breathing per minute (BPM) N1, and the CSI within first three seconds are discard, that is, a second time of breathing rate estimation is performed at 21st second to obtain a second BPM N2, and CSI from 3 seconds to 21 seconds are obtained for obtaining the breathing rate. Similarly, a third time of breathing rate estimation can be performed at 24th second to obtain a third BPM N3 by using CSI obtained from 6th second to 24th second, and the CSI obtained from 3rd second to 5th second can be discard, so as to update the data. Therefore, after the first time interval, the breathing rate can be updated once every three seconds, for example, and the present disclosure is not limited thereto.

Moreover, the filtered CSI can be further calculated by the median filter. For example, a set of CSI is obtained as an original data shown in the following Table III:

TABLE III

| Number | 1 | 2 | 3 | 4 | 5 | 6 | 7 | 8 | 9 | 10 | 11 | 12 | 13 | 14 | 15 | 16 | 17 |
|---|---|---|---|---|---|---|---|---|---|---|---|---|---|---|---|---|---|
| Original | 66 | 30 | 84 | 53 | 30 | 63 | 17 | 11 | 6 | 55 | 72 | 58 | 54 | 35 | 50 | 4 | 95 |
| data | | | | | | | | | | | | | | | | | |

In the present embodiment, original data may be amplitude data of one subcarrier within a time interval. The kernel size of the median filter can be set to 3, which means successively three of the original data are used to obtain a median as one entry of the filtered CSI. That is, a moving window with size of 3 can be utilized as the median filter to obtain the filtered CSI, as shown in the following Table IV:

TABLE IV

| Number | 1 | 2 | 3 | 4 | 5 | 6 | 7 | 8 | 9 | 10 | 11 | 12 | 13 | 14 | 15 | 16 | 17 |
|---|---|---|---|---|---|---|---|---|---|---|---|---|---|---|---|---|---|
| Data after | 66 | 53 | 53 | 53 | 30 | 17 | 11 | 11 | 55 | 58 | 58 | 54 | 50 | 35 | 50 | 57 | 70 |
| median | | | | | | | | | | | | | | | | | |
| filter | | | | | | | | | | | | | | | | | |

Therefore, after the CSI is filtered by the median filter, a new filtered set of amplitude data is created. Utilizing periodogram algorithm to obtain the breathing rate corresponding to the subcarrier. In light of median filter, noises formed by unreasonable values of CSI can be filtered, thereby reducing false estimations for the breathing rates.

In other preferred embodiments, the median filter can also be used if the results of the breathing rates are not available, so as to reduce valid detections.

In conclusion, the breath detection device and method thereof provided by the present disclosure can predict the one or more breathing rates by considering all subcarriers from the obtained CSI in the obtained parent set, analyze the results of the reaction of the subcarriers in the indoor environment according to the statistical distribution ratio, and select the breathing rates having the highest proportion to obtain a reasonable breathing rate.

Furthermore, the breath detection device and method thereof provided by the present disclosure can reduce breathing detection costs without selecting a better state of the subcarrier beforehand, so as to ignore interference to subcarriers and improve an accuracy of breath detection.

The disclosed system, device and method can be realized by a specialized system having a functional block diagram illustration of a hardware platform which includes user interface elements. The computer may be a general purpose computer or a special purpose computer. Both can be used to implement a specialized system for the present teaching. This computer may be used to implement any component of the techniques of vital sign detection and monitoring based on channel state information, as described herein. For example, the system in FIG. 8 may be implemented on a computer, via its hardware, software program, firmware, or a combination thereof.

Those skilled in the art will recognize that the present teachings are amenable to a variety of modifications and/or enhancements. For example, although the implementation of various components described above may be embodied in a hardware device, it may also be implemented as a software only solution—e.g., an installation on an existing server. In addition, the vital sign detection and monitoring based on channel state information as disclosed herein may be implemented as a firmware, firmware/software combination, firmware/hardware combination, or a hardware/firmware/software combination.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. A breath detection device, comprising:
a receiver configured to receive at least one signal from a multipath channel being impacted by breathing of at least one living being;
a processor; and
a memory communicatively coupled to the processor;
wherein the processor is configured to:
extract channel state information (CSI) within a first time interval for the multipath channel from the at least one wireless signal;
calculate one or more breathing rates based on the CSI within the first time interval;
delete one or more breathing rates exceeding a predetermined range from the one or more breath breathing rates;
group the remaining breathing rates to obtain one or more breathing rate groups;
select the breathing rate group having largest quantity; and
take the breathing rate of the selected breathing rate group as an estimated breathing rate.

2. The breath detection device according to claim 1, wherein the predetermined range ranges from a lower breathing rate to a higher breathing rate, and the first time interval is larger than a duration associated to the high breathing rate.

3. The breath detection device according to claim 2, wherein the processor is further configured to:
extract CSI within a second time interval for the multipath channel from the at least one wireless signal, wherein the second time interval is after the first time interval; and
update the CSI within the first time interval with the CSI within the second time interval.

4. The breath detection device according to claim 3, wherein the processor is further configured to, in response to the one or more breathing rates are unable to be calculated based on the CSI within the first time interval, process the CSI within the first time interval with Median filter to obtain a filtered CSI, and calculate the one or more breathing rates based on the the filtered CSI.

5. The breath detection device according to claim 1, wherein the at least one signal is transmitted in a bandwidth of 80 MHz.

6. The breath detection device according to claim 1, further comprising a server communicatively coupled to the receiver, wherein the server includes the processor, and the receiver is configured to transmit data of the received at least one wireless signal to the server.

7. The breath detection device according to claim 1, wherein the processor is further configured to process the CSI within the first time interval with a Median filter.

8. The breath detection device according to claim 7, wherein a kernal size of the Median filter is set to be N, in which N of data of the CSI within the first time interval are averaged to obtained filtered data, and N is an odd number larger than 2.

9. The breath detection device according to claim 1, wherein the one or more breathing rates are calculated based on amplitudes of a plurality of subcarriers of the CSI within the first time interval.

10. A breath detection method, comprising the following steps:
configuring a transmitter to transmit at least one signal;
configuring a receiver to receive the at least one signal from a multipath channel being impacted by breathing of at least one living being;
configuring a processor to:
extract channel state information (CSI) within a first time interval for the multipath channel from the at least one wireless signal;
calculate one or more breathing rates based on the CSI within the first time interval;
delete one or more breathing rates exceeding a predetermined range from the one or more breath breathing rates;
group the remaining breathing rates to obtain one or more breathing rate groups;
select the breathing rate group having largest quantity; and
take the breathing rate of the selected breathing rate group as an estimated breathing rate.

11. The breath detection method according to claim 10, wherein the predetermined range ranges from a lower breathing rate to a higher breathing rate, and the first time interval is larger than a duration associated to the high breathing rate.

12. The breath detection method according to claim 11, further comprising:
configuring the processor to:
extract CSI within a second time interval for the multipath channel from the at least one wireless signal, wherein the second time interval is after the first time interval; and
update the CSI within the first time interval with the CSI within the second time interval.

13. The breath detection method according to claim 12, further comprising:
configuring the processor to, in response to the one or more breathing rates are unable to be calculated based on the CSI within the first time interval, process the CSI within the first time interval with Median filter to obtain a filtered CSI, and calculate the one or more breathing rates based on the filtered CSI.

14. The breath detection method according to claim 10, wherein the at least one signal is transmitted in a bandwidth of 80 MHz.

15. The breath detection method according to claim 10, further comprising:
configuring an antenna controller of the transmitter to control a directional antenna of the transmitter to transmit the at least one wireless signal in a transmitting direction toward the living being.

16. The breath detection method according to claim 10, further comprising:
configuring the receiver to transmit data of the received at least one wireless signal to the server, wherein the server includes the processor.

17. The breath detection method according to claim 10, further comprising:
configuring the processor to process the CSI within the first time interval with a median filter.

18. The breath detection method according to claim 17, further comprising:
configuring the processor to set a kernal size of the Median filter to N; and
configuring the processor to average N of data of the CSI within the first time interval to obtained filtered data, wherein N is an odd number larger than 2.

19. The breath detection method according to claim 10, further comprising:
calculating the one or more breathing rates based on amplitudes of a plurality of subcarriers of the CSI within the first time interval.
